(19) United States
(12) Patent Application Publication
(10) Pub. No.: US 2006/0158639 A1
(43) Pub. Date: Jul. 20, 2006

(54) Eye tracker and pupil characteristic measurement system and associated methods

(76) Inventors: John Alfred Campin, Orlando, FL (US); Young K. Kwon, Oviedo, FL (US); Phuoc Khanh Nguyen, Winter Springs, FL (US); Haizhang Li, Orlando, FL (US); Gary Paul Gray, Orlando, FL (US)

Correspondence Address: JACQUELINE E. HARTT, PH.D;ALLEN, DYER, DOPPELT, MILBRATH & GILCHRIST, P.A., P.O. BOX 3791, ORLANDO, FL, 32802-3791, US

(21) Appl. No.: 11/252,191

(22) Filed: Oct. 17, 2005

Related U.S. Application Data
(63) Continuation-in-part of application No. 10/156,654, filed on May 28, 2002, now abandoned.
(60) Provisional application No. 60/676,353, filed on Apr. 29, 2005.

(51) Int. Cl. G01P 3/36; A61B 18/18

(52) U.S. Cl. 356/29; 606/10; 606/5

(57) ABSTRACT

Systems and methods for tracking eye movement includes directing an incident light beam onto each facet of a pyramidal prism to produce a plurality of beams that form a plurality of light spots, at least two of the light spots having different diameters. The prism is translatable to effect a change in spacing of the light spots. Intensities of light reflected from the light spots is used to retain the light spots upon a pupil/iris boundary. A relative intensity of the spots indicates a change in pupil size. A second light spot positioned on a predetermined eye sector can also be used to calculate a pupil characteristic and an environmental effect on light received from the eye.

### CROSS-REFERENCE TO RELATED APPLICATION

This application is a continuation-in-part of Ser. No. 10/156,654, filed May 28, 2002, entitled “Zoom Device for Eye Tracker Control System and Associated Methods,” the contents of which are incorporated hereinto by reference.

### FIELD OF THE INVENTION

The invention relates generally to eye tracking devices for ophthalmic laser surgical systems, and more particularly to such a device that has a zoom capability.

### BACKGROUND OF THE INVENTION

The use of lasers to erode a portion of a corneal surface is known in the art to perform corrective surgery. In the field of ophthalmic medicine, photorefractive keratectomy (PRK), phototherapeutic keratectomy (PTK), laser in situ keratomileus (LASIK), and laser epithelial keratomileusis (LASEK) are procedures for laser correction of focusing deficiencies of the eye by modification of corneal profile.

In these procedures, surgical errors due to application of the treatment laser during unwanted eye movement can degrade the refractive outcome of the surgery. The eye movement or eye positioning is critical since the treatment laser is centered on the patient's theoretical visual axis which, practically speaking, is approximately the center of the patient's pupil. However, this visual axis is difficult to determine, owing in part to residual eye movement and involuntary eye movement, known as saccadic eye movement. Saccadic eye movement is high-speed movement (i.e., of very short duration, 10-20 milliseconds, and typically up to 1° of eye rotation) inherent in human vision and is used to provide a dynamic scene to the retina. Saccadic eye movement, while being small in amplitude, varies greatly from patient to patient due to psychological effects, body chemistry, surgical lighting conditions, etc. Thus, even though a surgeon may be able to recognize some eye movement and can typically inhibit/restart a treatment laser by operation of a manual switch, the surgeon's reaction time is not fast enough to move the treatment laser in correspondence with eye movement.

A system for performing eye tracking has been described in U.S. Pat. Nos. 5,632,742; 5,752,950; 5,980,513; 6,302,879; and 6,315,773, which are commonly owned with the present application, and the disclosures of which are incorporated hereinto by reference. An eye tracking system is described using reflections from four tracking beams positioned on the pupil/iris boundary to track eye movement. This system presupposes treating an eye having a dilated pupil, and it would be beneficial to provide a system that can also track movement of an eye with an undilated pupil.

When a tracking beam is inside the pupil area, the sensor receives a maximum return signal, since the reflective coefficient of the pupil area is higher than that of the iris area. Thus when the tracking beam is in the iris area only, a minimum return signal is received. A middle level, comprising the average of the maximum and minimum return signals, indicates that the tracking spot is on the pupil/iris boundary.

If surgery is being performed on an undilated pupil, the pupil size can change during surgery, which will affect the return signals from the four tracking spots. The control system would then move the spot optics to retain the spots on the pupil/iris boundary.

However, a signal change can also be the result of external disturbances, such as a change in scattering characteristics from the ablated plume of tissue and the corneal surface during surgery. Therefore, it would be beneficial to provide a system for compensating for such external changes.

### SUMMARY OF THE INVENTION

The present invention provides an eye tracking method and system that is used in conjunction with a laser system for performing corneal correction and includes a zooming feature for changing a separation of light spots incident upon the eye, collectively called the probe beam.

In accordance with the present invention, a zooming mechanism for use in an eye tracking system is disclosed that, in a first embodiment, comprises a pyramidal prism having a plurality of reflective facets meeting at an apex, oriented so that the apex points along an optical axis. Means are provided for directing an incident light beam onto each facet of the prism. Each incident light beam is reflected away from the prism in a direction pointing toward the apex. The directing means is adapted to produce a plurality of reflected beams that, when incident upon a planar surface substantially normal to the optical axis, form a plurality of light spots arrayed about the optical axis.

A second embodiment of the zooming mechanism comprises a pyramidal transmissive prism that has a plurality of facets meeting at an apex, the apex pointing along an optical axis. Means are provided for directing an incident light beam onto each facet of the prism. Each incident light beam is refracted within the prism to form a refracted beam in a direction pointing toward the apex. When the plurality of refracted beams are incident upon a planar surface substantially normal to the optical axis, a plurality of light spots are formed that are arrayed about the optical axis.

In both embodiments, means are provided for translating the prism along the optical axis between a first position wherein the light spots are separated by a first spacing and a second position wherein the light spots are separated by a second spacing that is smaller than the first spacing. The light spots thereby, in a preferred embodiment, have a substantially equal size with the prism in the first and the second positions.

In a system incorporating the zoom mechanism of the present invention, a light source generates a modulated light beam, for example, in the near-infrared 905-nanometer wavelength region. An optical delivery arrangement including the zoom mechanism converts each laser modulation interval into the plurality of light spots, which are focused such that they are incident on a corresponding plurality of positions located on a boundary whose movement is coincident with that of eye movement. The boundary can be defined by two visually adjoining surfaces having different coefficients of reflection. The boundary can be a naturally occurring boundary (e.g., the iris/pupil boundary or the iris/sclera boundary) or a manmade boundary (e.g., an ink ring drawn, imprinted or placed on the eye, or a contrast-enhancing tack affixed to the eye). Energy is reflected from each of the positions located on the boundary receiving the light spots. An optical receiving arrangement detects the reflected energy from each of the positions. Changes in reflected energy at one or more of the positions is indicative of eye movement.

One aspect of the method of the present invention comprises a method for sensing eye movement. This method comprises the steps of directing a plurality of light beams onto a plurality of positions on a boundary defined by two adjoining surfaces of the eye to form a plurality of light spots. The two surfaces are selected to have different coefficients of reflection. Reflected energy from each of the plurality of positions is detected, wherein changes in the reflected energy at one or more of the positions is indicative of eye movement. In order to retain the light spots on the boundary, a size of a pattern formed by the plurality of light spots is adjusted on the plurality of positions. This adjustment, in a preferred embodiment, is performed without substantially changing a diameter of the individual light spots.

Another aspect of the present invention is directed to a system and method for tracking eye movement and pupil size. The system comprises a pyramidal prism that has a plurality of reflective or transmissive facets pointing in an upstream direction along an optical axis. Means are provided for directing an incident light beam onto each facet of the prism. Each incident light beam is acted upon by the prism so that the light beam proceeds in a downstream direction along the optical axis. The directing means are preferably adapted to produce a plurality of transmitted beams that, when incident upon a surface substantially normal to the optical axis, form a plurality of light spots arrayed about the optical axis. At least two of the light spots have different diameters.

Means are also provided for translating the prism along the optical axis between a first position wherein the light spots are separated by a first spacing and a second position wherein the light spots are separated by a second spacing that is smaller than the first spacing.

Additionally provided are means for receiving light reflected from each of the light spots and means, in signal communication with the light-receiving means, for calculating from an intensity of the received light a position of the light spots. Finally, means are provided for calculating a desired position for the prism-translating means and for directing the prism-translating means to position and retain the light spots upon a pupil/iris boundary of the eye. The calculating means are also adapted to calculate from a relative intensity of the received light from at least some of the plurality of spots a change in pupil size.

Another aspect of the invention is directed to a system for tracking eye movement and pupil size. The system comprises means for directing a plurality of first light spots about an optical axis that is substantially normal to an eye. Means are also provided for retaining the first light spots on a first predetermined eye sector, for tracking eye movement.

Means are provided for directing a second light spot substantially along the optical axis and for scanning the second light spot across a second predetermined eye sector. Light reflected from the second light spot is received, and a change in intensity of this light is used to calculate a pupil characteristic.

### BRIEF DESCRIPTION OF THE DRAWINGS

FIG. 1 is a block diagram of an eye movement tracking system in accordance with the present invention.

FIG. 2 is a block diagram of an optical arrangement for the focusing optics in the eye tracking system.

FIG. 3 is a block diagram of an optical arrangement for the focusing optics in the eye tracking system using a pyramidal zoom device.

FIG. 4 is a schematic diagram of a translatable reflective prism being used in a zoom mechanism in a first position.

FIG. 5 is a schematic diagram of the translatable reflective prism of FIG. 3 in a second position.

FIG. 6 is a schematic diagram of a translatable transmissive prism being used in a zoom mechanism in a first position.

FIG. 7 is a schematic diagram of the translatable transmissive prism of FIG. 5 in a second position.

FIG. 8 is a schematic diagram of another embodiment of the use of a pyramidal prism to retain tracking spots on the pupil/iris boundary.

FIG. 9 illustrates the four tracker spots having two different spot sizes, each beam having the same energy, projected onto a contracting pupil.

FIG. 10 is a graph of receiving signal versus pupil radius.

FIG. 11 illustrates tracking spots on the pupil/iris boundary and a fifth spot on the cornea.

FIG. 12 is a schematic diagram of a translatable pyramidal prism being used to position the tracking spots as in FIG. 11.

FIG. 13 illustrates tracking spots on the pupil/iris boundary, a fifth spot on the cornea, and a sixth spot on the iris.

FIG. 14 is a schematic diagram of a translatable pyramidal prism being used to position the tracking spots as in FIG. 13.

### DETAILED DESCRIPTION OF THE INVENTION

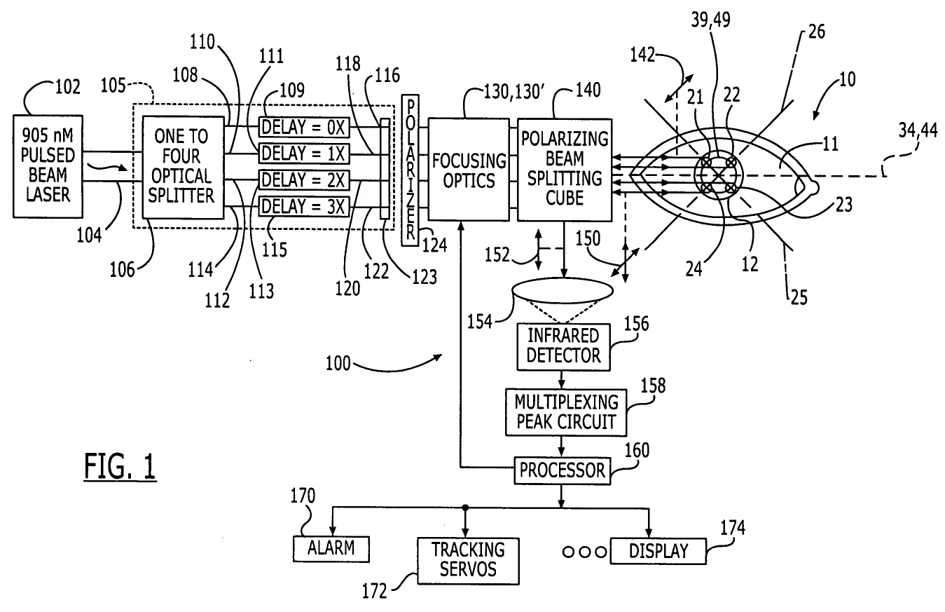

A description of a preferred embodiment of the present invention will now be presented with reference to FIGS. 1-14.

A preferred embodiment system, referenced generally by numeral 100, for carrying out the method of the present invention will now be described with the aid of the block diagram shown in FIG. 1. System 100 may be broken down into a delivery portion and a receiving portion. The delivery portion projects light spots 21, 22, 23, and 24 onto eye 10, while the receiving portion monitors reflections caused by light spots 21, 22, 23, and 24.

The delivery portion includes a laser 102 transmitting light through optical fiber 104 to an optical fiber assembly 105 that splits and delays each pulse from laser 102 into preferably four equal-energy pulses. An exemplary laser 102 comprises a 905-nanometer pulsed diode, although this is not intended as a limitation. Assembly 105 includes a one-to-four optical splitter 106 that outputs four pulses of approximately equal energy into optical fibers 108, 110, 112, 114. Such optical splitters are commercially available (e.g., model HLS2X4 manufactured by Canstar and model MMSC-0404-0850-A-H-1 manufactured by E-Tek Dynamics). In order to use a single processor to process the reflections caused by each pulse transmitted by fibers 108,110,112, and 114, each pulse is uniquely multiplexed by a respective fiber optic delay line (or optical modulator) 109, 111, 113, and 115. For example, delay line 109 causes a delay of zero, i.e., DELAY=Ox where x is the delay increment; delay line 111 causes a delay of x, i.e., DELAY=1x; etc.

The pulse repetition frequency and delay increment x are chosen so that the data rate of system 100 is greater than the speed of the movement of interest. In terms of saccadic eye movement, the data rate of system 100 must be on the order of at least several hundred hertz. For example, a system data rate of 4 kHz is achieved by (1) selecting a small but sufficient value for x to allow processor 160 to handle the data (e.g., 250 nanoseconds), and (2) selecting the time between pulses from laser 102 to be 250 microseconds (i.e., laser 102 is pulsed at a 4-kHz rate).

The four equal-energy pulses exit assembly 105 via optical fibers 116,118,120, and 122, which are configured as a fiber optic bundle 123. Bundle 123 arranges optical fibers 116,118,120, and 122 in a manner that produces a square (dotted line) with the center of each fiber at a corner thereof.

Light from assembly 105 is passed through an optical polarizer 124 that attenuates the vertical component of the light and outputs horizontally polarized light beams as indicated by arrow 126. Horizontally polarized light beams 126 pass to focusing optics 130, where the spacing between beams 126 is adjusted based on the boundary of interest. Additionally, a zoom capability can be provided to allow for adjustment of the size of the pattern formed by spots 21-24. This capability allows system 100 to adapt to different patients, boundaries, etc. In particular embodiments, the spots 21-24 are focused on a boundary between the iris and the sclera or on a boundary between the iris and the pupil.

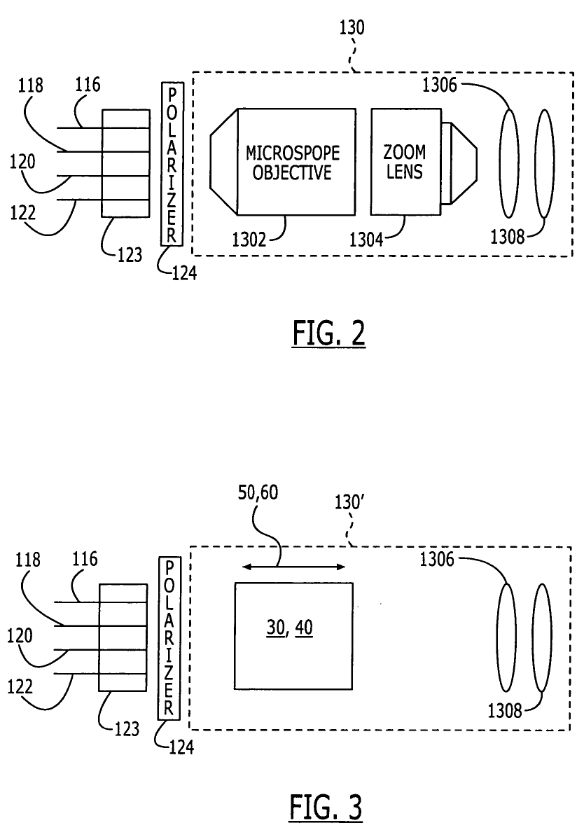

While a variety of optical arrangements are possible for focusing optics 130, one such arrangement is shown by way of example in FIG. 2. In FIG. 2, fiber optic bundle 123 is positioned at the working distance of microscope objective 1302. The numerical aperture of microscope objective 1302 is selected to be equal to the numerical aperture of fibers 116,118,120, and 122. Microscope objective 1302 magnifies and collimates the incoming light. Zoom lens 1304 provides an additional magnification factor for further tunability. Collimating lens 1306 has a focal length that is equal to its distance from the image of zoom lens 1304 such that its output is collimated. The focal length of imaging lens 1308 is the distance to the eye such that imaging lens 1308 focuses the light as four sharp spots on the corneal surface of the eye.

The zoom lens 1304 as described above changes the probe beam geometry, that is, the inscribed circle that contains all the probe beams, in order to accommodate varying object sizes and boundaries. A standard zoom lens 1304 may be used for this purpose; however, the dynamic range for laser tracking devices using standard zoom lenses is limited because the individual probe beam size is changed in direct proportion to the overall probe beam geometry.

In order to optimize dynamic range, the magnification of the overall probe beam geometry, that is, the inscribed circle of spots 21-24, would preferably be decoupled from that of the individual beam size. Two embodiments of a system and method for achieving such a decoupling will now be presented with reference to FIGS. 3-7, with FIG. 3 representing a block diagram of an optical arrangement for the focusing optics 130′ in the eye tracking system using a pyramidal zoom device.

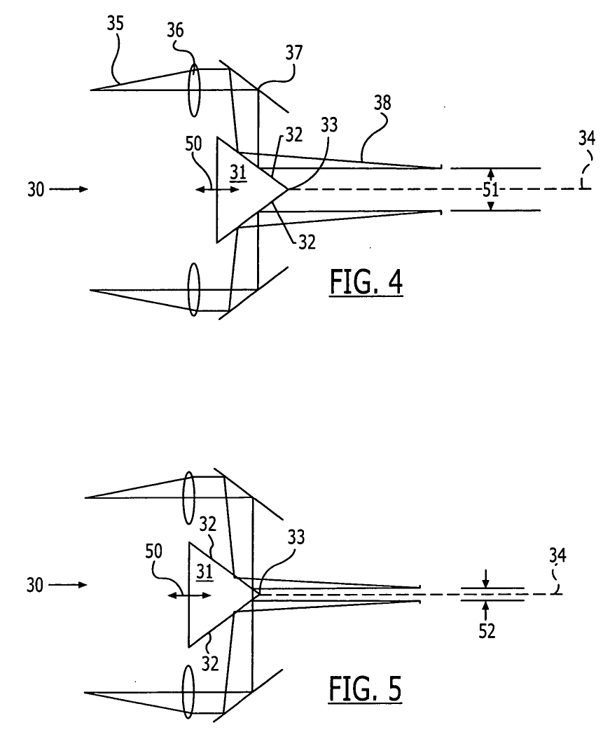

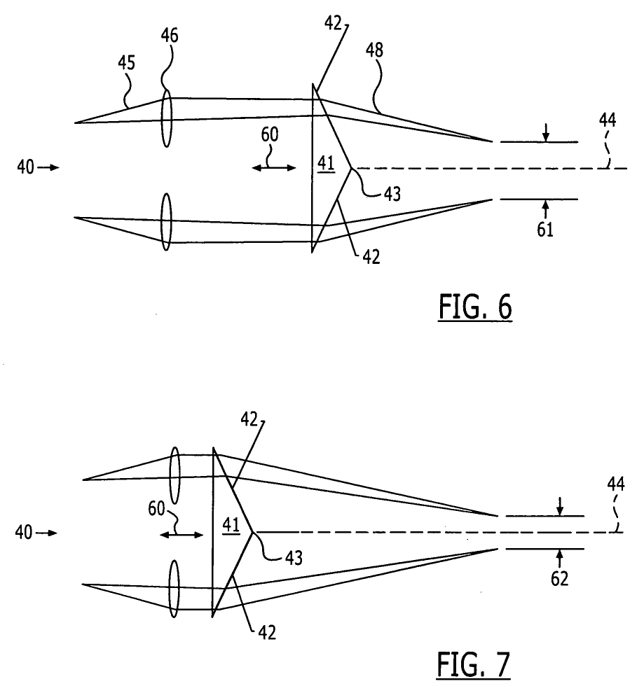

A first embodiment of the zoom mechanism 30 comprises a pyramidal prism 31 having a plurality of, in a preferred embodiment four, reflective facets 32 (FIGS. 4 and 5). It will be understood by one of skill in the art that FIGS. 4 and 5 (and subsequently discussed FIGS. 6 and 7) are highly schematic representations in two dimensions for ease of presentation, four-sided pyramidal prisms being well known in the art.

The facets 32 meet at an apex 33 that points along an optical axis 34. It will also be understood by one of skill in the art that by “apex” is meant herein the point or sector at which the facets reach their smallest dimension, and that the prism may in fact comprise a truncated pyramid without a pointed apex.

An incident light beam 35 is directed onto each facet 32 of the prism 31 by an optical arrangement comprising a focusing lens 36 that is positioned to receive an incident light beam 35 and is adapted to image the respective incident light beam 35 to an image plane.

In a preferred embodiment a generally planar mirror 37 is disposed in the optical pathway to receive the respective incident light beam 35 downstream of the respective focusing lens 36 and to reflect the respective incident light beam 35 onto a selected prism facet 32. Preferably the mirror 37 is oriented substantially parallel to the selected prism facet 32. The mirror 37 is present in a preferred embodiment to serve as a “folding” mirror for reducing a size of the mechanism 30.

Each incident light beam 35 is then reflected away from the prism 31 in a direction pointing toward the apex 33, producing a plurality of reflected beams 38. When the reflected beams 38 are incident upon a planar surface substantially normal to the optical axis 34 to form the plurality of light spots 21-24 (FIG. 1) arrayed substantially on an inscribed circle 39 about the optical axis 34 substantially in a square pattern.

A second embodiment of the zoom mechanism 40 comprises a pyramidal transmissive prism 41 having a plurality of, in a preferred embodiment four, facets 42 (FIGS. 6 and 7). The facets 42 meet at an apex 43 that points along an optical axis 44.

An incident light beam 45 is directed onto each facet 42 of the prism 41 by an optical arrangement comprising a focusing lens 46 that is positioned to receive an incident light beam 45 and is adapted to image the respective incident light beam 45 to an image plane.

Each incident light beam 45 refracted within the prism 41 to form a refracted beam 48 in a direction pointing toward the apex 43. The plurality of refracted beams 48, when incident upon a planar surface substantially normal to the optical axis 44, form the plurality of light spots 21-24 arrayed substantially in a square on an inscribed circle 49 (FIG. 1) about the optical axis 44.

The zooming mechanisms 30,40 further comprise a mechanism 50,60 for translating the prism 31,41 along the optical axis 34,44 between a first position (FIGS.4 and 6) wherein the light spots 21-24 are separated by a first spacing 51,61 and a second position (FIGS. 5 and 7) wherein the light spots 21-24 are separated by a second spacing 52,62 smaller than the first spacing 51,61. In this arrangement, the light spots 21-24 advantageously have a substantially equal size with the prism 31,41 in the first and the second positions. The translating mechanism 50,60 may comprise, for example, a motorized translating stage such as is known in the art that is under processor 160 control.

Referring again to FIG. 1, polarizing beam splitting cube 140 receives horizontally polarized light beams 126 from focusing optics 130. Polarization beamsplitting cubes are well known in the art. By way of example, cube 140 is a model 10FC16PB.5 manufactured by Newport-Klinger. Cube 140 is configured to transmit only horizontal polarization and reflect vertical polarization. Accordingly, cube 140 transmits only horizontally polarized light beams 126 as indicated by arrow 142. Thus it is only horizontally polarized light that is incident on eye 10 as spots 21-24. Upon reflection from eye 10, the light energy is depolarized (i.e., it has both horizontal and vertical polarization components), as indicated by crossed arrows 150. The vertical component of the reflected light is then directed/reflected as indicated by arrow 152. Thus cube 140 serves to separate the transmitted light energy from the reflected light energy for accurate measurement.

The vertically polarized portion of the reflection from spots 21-24 is passed through focusing lens 154 for imaging onto an infrared detector 156. Detector 156 passes its signal to a multiplexing peak detecting circuit 158, which is essentially a plurality of peak sample-and-hold circuits, a variety of which are well known in the art. Circuit 158 is configured to sample (and hold the peak value from) detector 156 in accordance with the pulse repetition frequency of laser 102 and the delay x. For example, if the pulse repetition frequency of laser 102 is 4 kHz, circuit 158 gathers reflections from spots 21-24 every 250 microseconds.

By way of example, infrared detector 156 is an avalanche photodiode model C30916E manufactured by EG&G. For a given transmitted laser pulse, the detector output will consist of four pulses separated in time by the delays associated with optical delay lines 109, 111, 113, and 115 shown in FIG. 1. These four time-separated pulses are fed to peak-and-hold circuits. Input enabling signals are also fed to the peak-and-hold circuits in synchronism with the laser fire command. The enabling signal for each peak and hold circuit is delayed by delay circuits. The delays are set to correspond to the delays of delay lines 109, 111, 113, and 115 to allow each of the four pulses to be input to the peak-and-hold circuits. The reflected energy associated with a group of four spots is collected as the detector signal is acquired by all four peak and hold circuits. At this point, an output multiplexer reads the value held by each peak-and-hold circuit and inputs them sequentially to processor 160.

The values associated with the reflected energy for each group of four spots (i.e., each pulse of laser 102) are passed to a processor 160, where horizontal and vertical components of eye movement are determined. For example, let R₂₁, R₂₂, R₂₃, and R₂₄ represent the detected amount of reflection from one group of spots 21-24, respectively. A quantitative amount of horizontal movement is determined directly from the normalized relationship

$$\frac{( {R}_{21} + {R}_{24} ) - ( {R}_{22} + {R}_{23} )}{{R}_{21} + {R}_{22} + {R}_{23} + {R}_{24}}$$

while a quantitative amount of vertical movement is determined directly from the normalized relationship

$$\frac{( {R}_{21} + {R}_{24} ) - ( {R}_{22} + {R}_{23} )}{{R}_{21} + {R}_{22} + {R}_{23} + {R}_{24}}$$

Note that normalizing (i.e., dividing by R₂₁+R₂₂+R₂₃+R₂₄) reduces the effects of variations in signal strength.

Once processed, the reflection differentials indicating eye movement (or the lack thereof can be used in a variety of ways. For example, an excessive amount of eye movement may be used to trigger an alarm 170. In addition, the reflection differential may be used as a feedback control for tracking servos 172 used to position an ablation laser. Still further, the reflection differentials can be displayed on display 174 for monitoring or teaching purposes.

Additionally, the detected reflected energy from light spots 21-24 may be analyzed in the processor 160 to determine a change in pupil size as determined by the reflection differentials and the spacing of the light spots 21-24. As it is desired to retain the light spots 21-24 on a selected eye surface boundary, here coincident with the circle 39,49, means are provided under direction of the processor 160 for directing the translating mechanism 50,60 to translate the prism 31,41 in a direction for retaining the light spots 21-24 on the selected boundary 39,49, without substantially altering the diameters of the light spots 21-24.

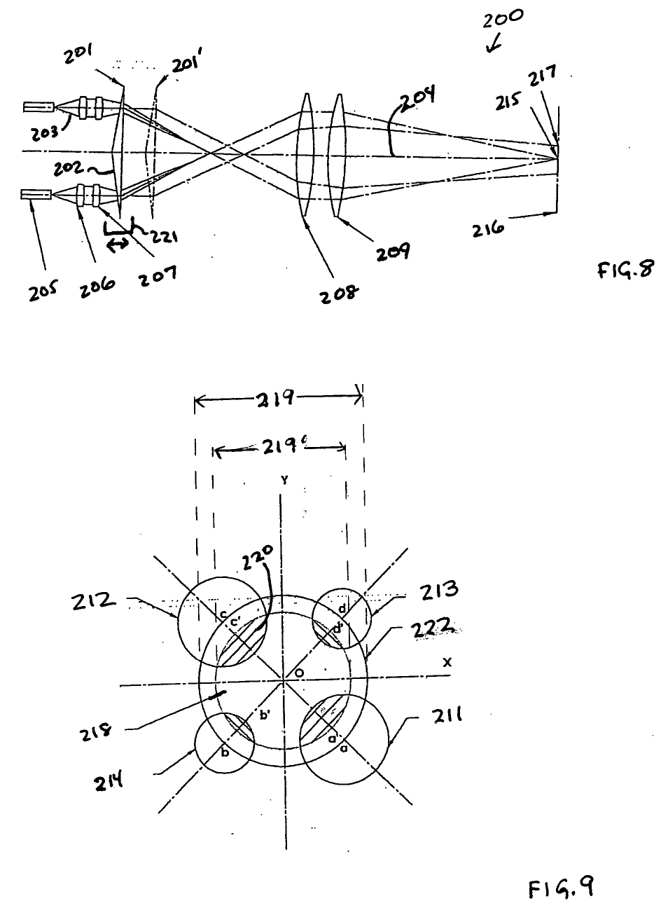

Another aspect of the present invention is directed to a tracker system 200 (FIGS. 8-10) for tracking both eye movement and pupil contraction and dilation, for use, for example, during refractive eye surgery on an undilated eye, although this is not intended as a limitation. In a particular embodiment, the system 200 comprises a pyramidal prism 201 that has a plurality of reflective or transmissive facets, shown in FIG. 8 as four transmissive facets 202 (two are shown) that act upon each incoming beam 203 to cause the beam 203 to proceed in an upstream direction along an optical axis 204.

The incident light beams 203 are directed onto the prism's facets 202 from, in a particular embodiment, a single pulsed laser emitting, for example, in the infrared, which is split into four substantially equal-energy pulses, and are delayed as described above. Each of the four pulses is directed onto an optical fiber 205, two of which are shown in FIG. 8, emerging beams 203 from which are collimated by a fiber lens 206 and then are sent through a first relay lens 207, which directs the beams 203 onto their respective prism facet 202. Each incident light beam is transmitted through the prism 201 so as to point in a downstream direction along the optical axis 204.

After emerging from the prism 201 the beams 203 are collimated by a unitary second relay lens 208, and then pass through a unitary imaging lens 209. The beams 203 are then directed so as to be incident upon a surface substantially normal to the optical axis 204, forming a plurality of light spots, here four light spots 211-214 that are arrayed about the optical axis 204 (FIG. 9). As described above, a translation of the prism 201 along the optical axis 204 causes the spots 211-214 to move radially relative to the optical axis 204. In FIG. 8, when the prism 201 is in the first position (solid line), the tracking spots 211-214 are overlapped at the center 215 of the eye plane 216; when the prism 201 is moved to a second position (dotted line), the spots 211-214 do not overlap, and impinge on the eye plane 216 in spaced-apart relation from each other at position 217. Thus, by moving the pyramidal prism 201, pupil size change can be accommodated.

In the present system 200, at least two of the light spots have different diameters. Here, beams 211,212 have larger areas than do beams 213,214, but have substantially equal total energy. In FIG. 9 a pupil 218 is shown contracting from a first diameter 219 to a second diameter 219′. The hatched areas 220 are the areas of the pupil 218 that are common in both pupil sizes.

As described above with reference to FIG. 1, a detector 156 is provided for receiving light reflected from each of the light spots 211-214 and processor means 160, in signal communication with the light receiver 156, for calculating from an intensity of the received light a position of the light spots. The tracking sampling speed can be, for example, as high as 4000 Hz.

Also as described above, means are provided for calculating a desired position for the prism 201 and for directing prism-translating means 221 to position and retain the light spots 211-214 upon a pupil/iris boundary 222 of the eye.

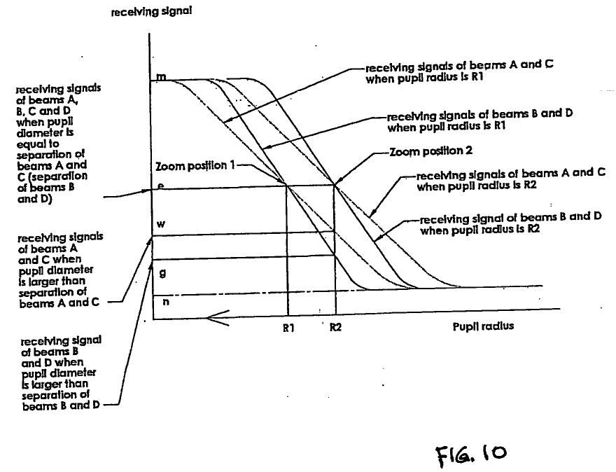

A method for detecting pupil size changes will now be discussed with reference to FIGS. 9 and 10, wherein beams 211-214 are illustrated as being positioned on the pupil/iris boundary 222 and having different overlaps depending upon pupil size.

The receiving signal has its maximum and minimum values when the tracking beams are totally inside and outside the pupil 218, respectively. When the pupil size changes, the receiving signal for the smaller beams changes more than the that from the larger beams. This is owing to the fact that the smaller beam has a steeper slope in the transitional area that does the larger beam. As shown in FIG. 10, the receiving signal change for the smaller beams are plotted with solid lines, while the receiving signal change for the larger beams are plotted with dashed lines. Considering a reference state when the receiving signal level of beams 211-214 are all equal (indicated by e), the radius of the pupil is indicated as R1, and the corresponding prism position is denoted as “zoom position I.”

When the pupil 218 contracts, the radius becomes R2, and the receiving signal for the larger beams (indicated as w) reduces less than that of the smaller beams (indicated as g). One can then use the signal difference between w and g as an indicator of pupil radius change from the reference point e. By moving the prism 201, one can drive four beam spots 211-214 so that the receiving signal of the beams becomes equal again (indicated as “zoom position II”). Here, beams A-D comprise beams 211-214, respectively, and the difference (A+C)−(B+D) provides an indication of a change in pupil size.

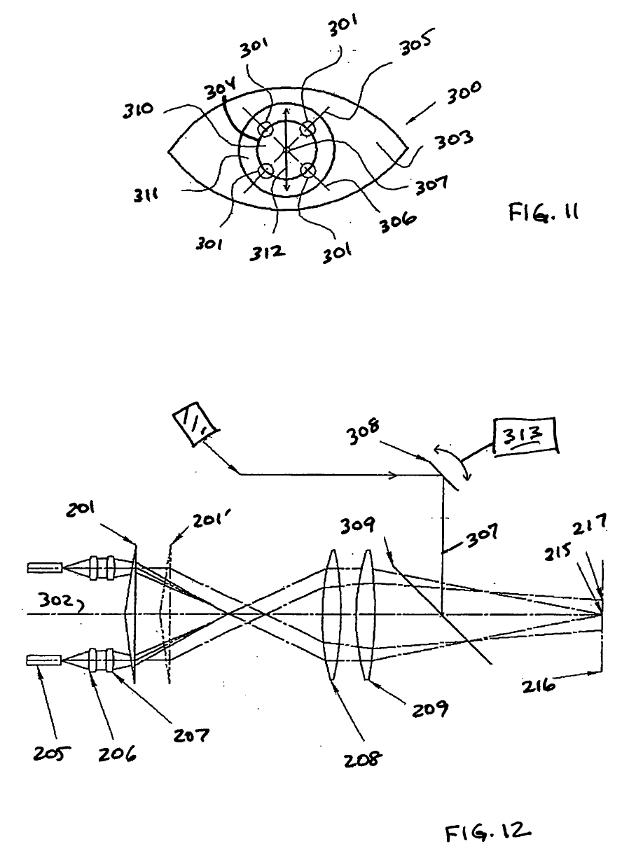

Another aspect of the invention is directed to systems 300,350 for tracking eye movement and pupil size (FIGS. 11-14), which can also be used, for example, during laser refractive surgery on an undilated pupil. As described above, tracking beams 21-24 or 211-214 are used to gather reflected light, an analysis of which provides data on eye position.

The system 300 in an exemplary first subembodiment (FIGS. 11 and 12) comprises means for directing a plurality of first light spots 301 about an optical axis 302 that is substantially normal to an eye 303. Means are also provided for retaining the first light spots 301 on a first predetermined eye sector, for tracking eye movement. As shown in FIG. 11, an exemplary set of first light spots 301 comprise four first light spots arrayed about the iris/pupil boundary 304 in a substantially square array and situated on a first axis 305 and a second axis 306 substantially orthogonal to the first axis 305. The systems and methods for using these four spots 301 to perform eye tracking is described above in exemplary embodiments with reference to FIGS. 1-7, and will not be repeated here.

The four-beam tracker as described, however, cannot discriminate among signal changes caused by external disturbances, such as changes in scattering characteristics from an ablated plume and the corneal surface during surgery, versus changes owing to pupil contraction/dilation.

A second type of light spot 307 is directed substantially along the optical axis 302, for example, with the use of a scanning mirror 308 and beam combiner 309 (FIG. 12). The scanning mirror 308 is adapted to scan light spot 307 across a second predetermined eye sector, for example, the pupil 310 and iris 311. The scanning can be directed, for example, along a third axis 312 in angular spaced relation from the first 305 and the second 306 axes, here shown as a vertical axis, with axes 305,306 approximately 45° from the vertical. The scanning mirror 308 can be driven, for example, by a piezo actuator 313, to steer the light spot 307 between the pupil area and the iris area. Light reflected from the second type of light spot 307 is received, and a change in intensity of this light is used to calculate a pupil characteristic and provide a baseline maximum return signal when positioned on the pupil 310 and a baseline minimum return signal when positioned on the iris 311. As an example, data from the received light can be used to calculate a pupil size and/or pupil center at a high frequency during laser surgery. Preferably, the beams are all separated by time delays to permit discrimination among them.

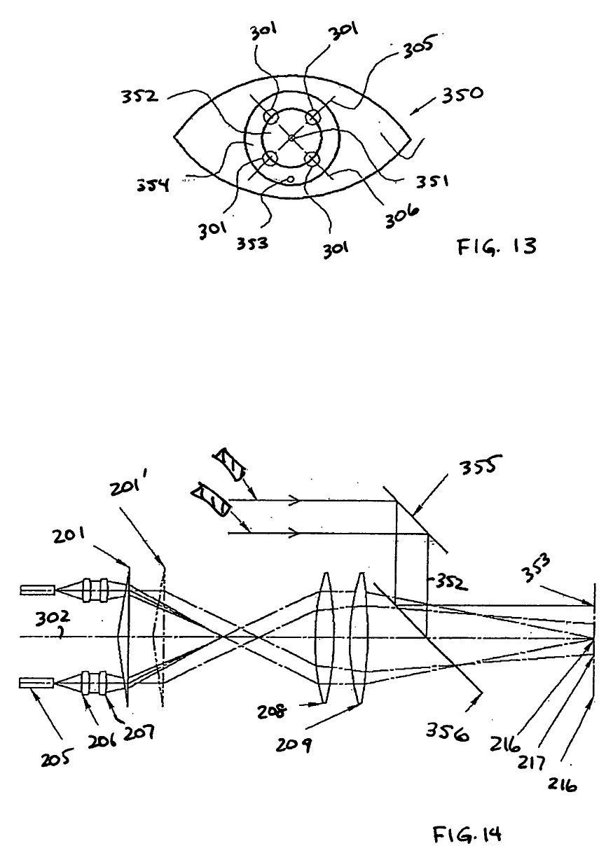

In a second subembodiment 350 to that above (FIGS. 13 and 14), the second type of light spot 351 is directed to the pupil 352, and a third type of light spot 353 is directed to the iris 354. The second type of light spot 351 is directed substantially along the optical axis 302, for example, with the use of a mirror 355 and beam combiner 356 (FIG. 14). The mirror 355 and beam combiner 356 are adapted to direct light spots 352,353 onto a second and a third predetermined eye sector, for example, the pupil 352 and iris 354, respectively. Light reflected from the second 351 and third 353 types of light spot is received, the intensities of which are used to compensate for changes in an environmental and/or an ocular characteristic, since the return signal from the pupil 310 can be used as a baseline maximum, and that from the iris 311, as a baseline minimum.

The advantages of the present invention are numerous. Eye movement and pupil and iris characteristics are sensed in accordance with a non-intrusive method and apparatus. The present invention will find great utility in a variety of ophthalmic surgical procedures without any detrimental effects to the eye or interruption of a surgeon's view, permitting surgical procedures to be performed on an undilated eye, for example. Further, data rates needed to sense saccadic eye movement are easily and economically achieved.

Although the invention has been described relative to a specific embodiment thereof, there are numerous variations and modifications that will be readily apparent to those skilled in the art in the light of the above teachings. It is therefore to be understood that, within the scope of the appended claims, the invention may be practiced other than as specifically described.

What is claimed is:

1. A system for tracking eye movement and pupil size comprising:
a pyramidal prism having a plurality of facets pointing in an upstream direction along an optical axis, the facets one of transmissive and reflective;
means for directing an incident light beam onto each facet of the prism, each incident light beam acted upon by the prism so as to cause the light beam to proceed in a downstream direction along the optical axis, the directing means adapted to produce a plurality of beams that, when incident upon a surface substantially normal to the optical axis, form a plurality of light spots arrayed about the optical axis, at least two of the light spots having different diameters;
means for translating the prism along the optical axis between a first position wherein the light spots are separated by a first spacing and a second position wherein the light spots are separated by a second spacing smaller than the first spacing;
means for receiving light reflected from each of the light spots;
means in signal communication with the light-receiving means for calculating from an intensity of the received light a position of the light spots;
means for calculating a desired position for the prism-translating means and for directing the prism-translating means to position and retain the light spots upon a pupil/iris boundary of an eye; and
means for calculating from a relative intensity of the received light from at least some of the plurality of spots a change in pupil size.

2. The system recited in claim 1, wherein each of the light spots has a substantially equal respective size with the prism in the first and the second positions.

3. The system recited in claim 1, further comprising a lens system downstream of the prism for focusing the light spots onto the pupil/iris boundary.

4. The system recited in claim 3, wherein the lens system comprises a relay lens downstream of the prism and an imaging lens downstream of the relay lens and upstream of the pupil/iris boundary.

5. The system recited in claim 1, wherein the incident-light-beam directing means comprises a plurality of optical trains, each optical train disposed to receive the respective incident light beam upstream of the prism and to direct the respective incident light beam onto a unitary prism facet.

6. The system recited in claim 5, wherein each optical train comprises a fiber lens positioned to receive and collimate a light beam from an optical fiber and a relay lens positioned to receive the light beam collimated by the fiber lens and to transmit the collimated light beam to the respective prism facet.

7. The system recited in claim 1, wherein the plurality of facets comprise four facets, the incident light beam comprises four light beams, and the plurality of light spots comprise four light spots having geometrical centers arrayed substantially in a square pattern.

8. The system recited in claim 1, wherein the means for calculating a change in pupil size comprises means for calculating a difference in an intensity of the received light from a first light spot and from a second light spot larger than the first light spot, the difference in intensity indicative of a change in pupil size.

9. A system for tracking eye movement comprising:
means for directing a plurality of first light spots about an optical axis substantially normal to an eye and for retaining the first light spots on a first predetermined eye sector, for tracking eye movement;
means for directing a second light spot substantially along the optical axis onto a second predetermined eye sector;
means for receiving light reflected from the second light spot; and
means in signal communication with the light-receiving means for calculating from a change in intensity of the received light at least one of a pupil characteristic and an environmental effect on light received from the eye.

10. The system recited in claim 9, wherein:
the plurality of first light spots comprise four light spots;
the first predetermined eye sector comprises a pupil/iris boundary; and
the second predetermined eye sector comprises at least one of a pupil and an iris.

11. The system recited in claim 10, further comprising means for scanning the second light spot across the pupil, and wherein the pupil characteristic comprises a pupil diameter.

12. The system recited in claim 9, wherein:
the second light spot comprises a pupil light spot directed to the pupil and an iris light spot directed to the iris;
the receiving means receives return signals from both the pupil light spot and the iris light spot; and
the calculating means determines an environmental effect using the return signals and transmits the environmental effect to the retaining means.

13. The system recited in claim 12, wherein the environmental effect comprises a light-transmissive change in a path between the eye and the receiving means.

14. A method for tracking eye movement and pupil size comprising the steps of:
directing an incident light beam onto each facet of a pyramidal prism having a plurality of facets pointing in an upstream direction along an optical axis, the facets one of transmissive and reflective, each incident light beam acted upon by the prism so as to cause the light beam to proceed in a downstream direction along the optical axis, in order to produce a plurality of beams that, when incident upon a surface substantially normal to the optical axis, form a plurality of light spots arrayed about the optical axis, at least two of the light spots having different diameters;
translating the prism along the optical axis between a first position wherein the light spots are separated by a first spacing and a second position wherein the light spots are separated by a second spacing smaller than the first spacing;
receiving light reflected from each of the light spots;
calculating from an intensity of the received light a position of the light spots;
calculating a desired position for the prism-translating means and translating the prism to position and retain the light spots upon a pupil/iris boundary of an eye; and
calculating from a relative intensity of the received light from at least some of the plurality of spots a change in pupil size.

15. The method recited in claim 14, wherein each of the light spots has a substantially equal respective size with the prism in the first and the second positions.

16. The method recited in claim 14, further comprising the step of directing light between the prism and the eye through a lens system for focusing the light spots onto the pupil/iris boundary.

17. The method recited in claim 14, wherein the incident-light-beam directing step comprises directing the incident light beam through a plurality of optical trains, each optical train disposed to receive the respective incident light beam upstream of the prism and to direct the respective incident light beam onto a unitary prism facet.

18. The method recited in claim 14, wherein the step of calculating a change in pupil size comprises calculating a difference in an intensity of the received light from a first light spot and from a second light spot larger than the first light spot, the difference in intensity indicative of a change in pupil size.

19. A method for tracking eye movement comprising the steps of:
directing a plurality of first light spots about an optical axis substantially normal to an eye;
retaining the first light spots on a first predetermined eye sector, for tracking eye movement;
directing a second light spot substantially along the optical axis onto a second predetermined eye sector;
receiving light reflected from the second light spot; and
calculating from a change in intensity of the received light at least one of a pupil characteristic and an environmental effect on light received from the eye.

20. The method recited in claim 19, wherein:
the plurality of first light spots comprise four light spots;
the first predetermined eye sector comprises a pupil/iris boundary; and
the second predetermined eye sector comprises at least one of a pupil and an iris.

21. The method recited in claim 20, further comprising the step of scanning the second light spot across the pupil, and wherein the pupil characteristic comprises a pupil diameter.

22. The method recited in claim 19, wherein:
the second light spot comprises a pupil light spot directed to the pupil and an iris light spot directed to the iris;
the receiving step comprises receiving return signals from both the pupil light spot and the iris light spot; and
the calculating step comprises determining an environmental effect using the return signals and further comprising using the environmental effect to perform the retaining step.

23. The method recited in claim 22, wherein the environmental effect comprises a light-transmissive change in a path between the eye and the receiving means.
